(19) United States
(12) Patent Application Publication
(10) Pub. No.: US 2014/0039632 A1
(43) Pub. Date: Feb. 6, 2014

(54) GLENOID VAULT FIXATION SYSTEM

(71) Applicant: IMDS Corporation, (US)

(72) Inventor: Michael Chad Hollis, Collierville, TN (US)

(73) Assignee: IMDS CORPORATION, Logan, UT (US)

(21) Appl. No.: 13/747,658

(22) Filed: Jan. 23, 2013

Related U.S. Application Data
(60) Provisional application No. 61/589,730, filed on Jan. 23, 2012.

(51) Int. Cl. A61F 2/40

(52) U.S. Cl. CPC A61F 2/4081
USPC 623/19.11

(57) ABSTRACT

A glenoid vault fixation device includes a body and at least one blade anchor. Interchangeable glenoid articular components connect to the body. The set of interchangeable components may address a continuum of glenoid and/or total shoulder indications. The body may provide multiple attachment points for the blade anchor, or may accept multiple anchors.

### CROSS-REFERENCE TO RELATED APPLICATIONS

This application claims the benefit of:

U.S. Application No. 61/589,730, filed Jan. 23, 2012, entitled GLENOID VAULT FIXATION SYSTEM, Attorney's docket no. MLI-109 PROV, which is pending.

The above-referenced document is hereby incorporated by reference in its entirety.

This application incorporates by reference the following application in its entirety:

U.S. patent application Ser. No. 12/640,892, filed Dec. 17, 2009, entitled INTERVERTEBRAL IMPLANT WITH INTEGRATED FIXATION, Attorney's docket no. SPINE 3.0-546 III, which is pending.

### BACKGROUND

The present disclosure includes an embodiment of a fixation device that may be used for glenoid joint reconstruction. Specifically, the embodiment described may be used for glenoid vault reconstruction, for example, due to cavitary or non-cavitary bone loss. It will be appreciated that the disclosed embodiment may have application outside of glenoid vault reconstruction applications, and may be used in reconstruction of other joints or tissue junctions in the body.

An example of the present technology is concerned with fusion of the glenoid vault by bladed anchors that extend outward from a central disc-like body. The bladed anchors may be secured to the body by dovetail features shaped to fit grooves on the body

### SUMMARY

In an aspect of the technology, a system includes a body having a first surface, an opposite second surface, and a first channel, wherein the first channel extends through the first and second surfaces, wherein the first channel includes a discrete first enlargement of a width of the first channel; and a first anchor coupled to the body, wherein the first anchor includes a first rail portion, a first blade portion, and a first intermediate portion connecting the first rail and first blade portions together, wherein the first rail portion and the first enlargement have complementary interlocking shapes, wherein the first rail portion is fixed within the first enlargement, the first intermediate portion occupies at least a portion of the first channel, and the first blade portion extends from the first surface of the body.

In an embodiment, the first channel includes a second discrete enlargement of the width of the first channel, wherein the first and second enlargements are periodically spaced along a length of the first channel, wherein the first rail portion and the second enlargement have complementary interlocking shapes.

In another embodiment, the second enlargement is shaped to receive the first blade portion.

In yet another embodiment, wherein the body includes a second channel spaced apart from the first channel, wherein the second channel includes a third discrete enlargement of a width of the second channel.

In yet another embodiment, the first rail portion and the third enlargement have complementary interlocking shapes.

In yet another embodiment, a second anchor is coupled to the body, wherein the second anchor includes a second rail portion, a second blade portion, and a second intermediate portion connecting the second rail and second blade portions together, wherein the second rail portion and the third enlargement have complementary interlocking shapes, wherein the second rail portion is fixed within the third enlargement, the second intermediate portion occupies at least a portion of the second channel, and the second blade portion extends from the first surface of the body.

In another aspect of the technology, a system includes a body, wherein the body includes a first surface, an opposite second surface, and a first connection feature; and a first anchor coupled to the body at the first connection feature so that a portion of the first anchor protrudes from the first surface.

In an embodiment, the system includes a second anchor; wherein the body includes a second connection feature; wherein the second anchor is coupled to the body at the second connection feature so that a portion of the second anchor protrudes from the first surface.

In another embodiment, the first and second anchors are interchangeably couplable to the body at the first and second connection features.

In yet another embodiment, the first and second anchors each include a blade portion, a rail portion, and an intermediate portion connecting the rail and blade portions together, wherein the rail portions of the first and second anchors are coupled to the body at the first and second connection features, respectively, and the blade portions of the first and second anchors protrude from the first surface.

In yet another embodiment, the blade portions of the first and second anchors converge.

### BRIEF DESCRIPTION OF THE DRAWINGS

While exemplary embodiments of the present technology have been shown and described in detail below, it will be clear to the person skilled in the art that changes and modifications may be made without departing from its scope. As such, that which is set forth in the following description and accompanying drawings is offered by way of illustration only and not as a limitation. The actual scope of the invention is intended to be defined by the following claims, along with the full range of equivalents to which such claims are entitled.

In addition, one of ordinary skill in the art will appreciate upon reading and understanding this disclosure that other variations for the technology described herein can be included within the scope of the present technology.

In the following Detailed Description, various features are grouped together in several embodiments for the purpose of streamlining the disclosure. This method of disclosure is not to be interpreted as reflecting an intention that exemplary embodiments of the technology require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

Identical reference numerals do not necessarily indicate an identical structure. Rather, the same reference numeral may be used to indicate a similar feature or a feature with similar functionality. Not every feature of each embodiment is labeled in every figure in which that embodiment appears, in order to keep the figures clear. Similar reference numbers (e.g., those that are identical except for the first numeral) are used to indicate similar features in different embodiments.

FIG. 1 is an isometric view of a glenoid fixation device; and

FIG. 2 is an isometric view of the glenoid fixation device of FIG. 1 with a scapula and a clavicle.

### DETAILED DESCRIPTION

Standard medical planes of reference and descriptive terminology are employed in this specification. A sagittal plane divides a body into right and left portions. A mid-sagittal plane divides the body into equal right and left halves. A coronal plane divides a body into anterior and posterior portions. A transverse plane divides a body into superior and inferior portions. Anterior means toward the front of the body. Posterior means toward the back of the body. Superior means toward the head. Inferior means toward the feet. Medial means toward the midline of the body. Lateral means away from the midline of the body. Axial means toward a central axis of the body. Abaxial means away from a central axis of the body.

The present technology may be employed in a glenoid fixation device which may be included in a system of implant components to address a continuum of glenoid conditions. The present technology may provide firm initial implant fixation and satisfactory pull-out strength while requiring minimal bone removal or resection compared to, for example, screw fixation. The present technology, which relies upon straight-line sliding engagement of fasteners, may be ergonomically and/or technically less demanding than screw fixation. In situ assembly of implant components may be advantageous in minimally invasive surgical procedures.

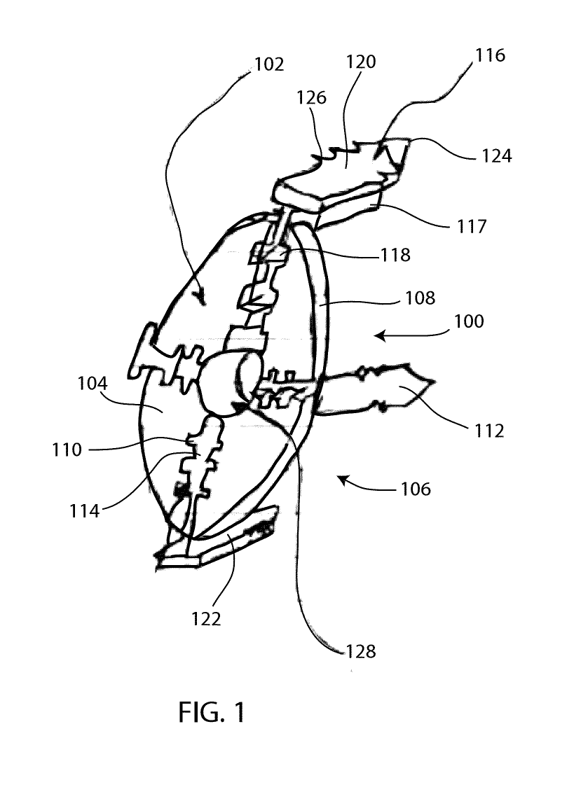

Referring to FIG. 1, a glenoid fixation device 100 includes a central body 102 and at least one anchor 112. The glenoid fixation device 100 may optionally include one or more of the following: screws, pegs, porous metal, and sutures.

Body 102 may be disc-shaped, oval, elliptical, polygonal, cruciate, asymmetrical, or irregular in profile. The body may include a top surface 104 and a bottom surface 106 opposite the top surface. The body may also include a first side surface 108 that extends between the top surface 104 and the bottom surface 106. First side surface 108 may extend circumferentially around at least a portion of the body 102. Side 108 may be referred to as a perimeter surface, and may extend around the entire body 102. A body center axis may be defined normal to (perpendicular to) the top surface 104 or bottom surface 106 at a centroid of the body.

The body 102 includes at least one channel 114 that extends through the top and bottom surfaces 104, 106. The channel 114 may extend between an interior region of the body and an outer region of the body. The channel 114 may intersect or break through the first side surface 108, or the first side surface 108 may remain intact. The channel 114 includes at least one cross slot, groove, or discrete enlargement 110 of the width of the channel. The cross slot 110 may be shaped for complementary fit with at least a portion of an anchor 112, fin, wing, blade, or other means for securing the body 102 to the bones of the glenoid vault. The cross slot 110 may be referred to as an anchor connection feature. The channel 114 may include plural discrete enlargements 110 periodically spaced along the length of the channel. The body may also include more than one channel 114.

FIG. 1 shows a body 102 with four evenly spaced channels 114 radiating from a central location. Each channel includes two cross slots 110, and two of the channels include a third cross slot. This arrangement may be appropriate when the body 102 is elliptical, so that longer channels with three cross slots are oriented along a major axis of the body, and shorter channels with two cross slots are oriented along a minor axis of the body. This arrangement may also be appropriate in order to provide wider spacing between cross slots along the channels with two cross slots and narrower spacing between cross slots along the channels with three cross slots. In other arrangements, there may be a different number of channels radiating outwardly from a central location. The channels may have varying numbers of cross slots. In yet other arrangements, the channels may lie tangential to the central location instead of radiating from the central location.

The body may also include a means for attaching another implant component, such as interchangeable glenoid articulating components, augment components, glenosphere components, metaglene components, or fusion components. The implant component attachment means 128 may be a female Morse taper 128. Other recessed and/or protruding connection features may be substituted, such as protruding Morse taper, internal or external threads, dovetails, undercuts, snap features, rails, and the like. The body may also contain at least one aperture, fenestration, or void (not shown) that may allow for the insertion of bone growth stimulation agents, such as osteoinductive and osteoconductive materials. The fenestration may be located in the central and/or outer region of the body. In one example, a fenestration may be present between each adjacent pair of channels 114. In another example, unoccupied void space within the channel(s) and/or cross slot(s) 110 may be packed with bone growth stimulating material.

The anchor 112 includes a blade portion 116 and a rail portion 118 connected together by an intermediate portion 117. In an end view, the width of the blade portion 116 and rail portion 118 is larger than the width of the intermediate portion 117. For example, FIG. 1 shows an anchor 112 which resembles a capital letter “H,” with the blade portion 116 forming a first upright of the “H,” the rail portion 118 forming a second upright of the “H,” and the intermediate portion 117 forming a crossbar of the “H.” In this example, width is along the uprights, or perpendicular to the crossbar.

The blade portion 116 includes a first surface 120 and a second surface 122 opposite the first surface 120. When the anchor 112 is attached to the body 102, the first surface 120 of the blade portion 116 may face away from the body 102 and the second surface 122 may face toward the body 102. The blade portion 116 may include a sharpened point 124 or edge to facilitate entrance of the anchor 112 into a bone or other tissue. The blade portion 116 may also include texture such as teeth 126, roughening, or ridges to enhance fixation of the anchor 112 in the bone.

The intermediate portion 117 extends between the blade portion 116 and the rail portion 118, and may protrude from the second surface 122. The length of the intermediate portion may be greater than, equal to, or less than the length of the blade portion 116. The intermediate portion 117 may include a sharpened leading edge to facilitate entrance of the anchor 112 into a bone or other tissue.

The rail portion 118 extends from the intermediate portion 117 opposite the blade portion 116. The length of the rail portion may be greater than, equal to, or less than the length of the blade portion 116, the intermediate portion 117, and/or the thickness of the body 102. FIG. 1 shows an example in which the length of the rail portion 118 is less than the length of the blade portion 116 and the intermediate portion 117, and is about equal to the thickness of the body 102. The rail portion 118 is shaped to fit into the enlargement 110 on the body 102. The rail portion 118 and the enlargement 110 may have geometrically complementary interlocking shapes, and may be sized for an interference fit, a line-to-line fit, or a clearance fit. The rail portion 118 establishes an anchor longitudinal axis which extends parallel to the direction the rail portion 118 slides into the enlargement 110.

The anchors in the present disclosure may share some or all of the features of the anchors disclosed in pending U.S. patent application Ser. No. 12/640,892 to Bae, et al., which is incorporated by reference herein in its entirety.

The anchor 112 may be attached to the body 102 by sliding the rail 118 into the cross slot 110 parallel to the anchor longitudinal axis such that the intermediate portion 117 occupies at least a portion of the channel 114 and the blade 116 extends from the bottom surface 106 of the body 102. The body 102 and/or anchor 112 may include mutual locking features to prevent migration or unintentional removal of the anchor 112 from the body 102. As the rail 118 slides forward into full engagement with the cross slot 110, mutual locking features may prevent the rail from sliding farther in the cross slot, and may also prevent the rail from sliding backwards out of the cross slot.

The number and location of the channels 114 and cross slots 110 may provide several choices of attachment location for the anchor 112 on the body 102. Where multiple anchors are used, the anchors may be interchangeably coupled to the body at any attachment location, such that the anchors 112 may be adjusted, for example, inwards and outwards on the body.

FIG. 1 shows an example in which the anchor 112 placement may be variable in the superior-inferior as well as the anterior-posterior directions to accommodate variations in the glenoid anatomy. Each anchor 112 may be independently radially positioned relative to the body 102.

In one example, the size and spacing between cross slots 110 along a channel 114 may complement the width and height of the anchor blade portion 116 and rail portion 118. An anchor rail 118 may thus be complementarily received in a first cross slot 110 and the anchor blade 116 may be received in an adjacent second cross slot of the same channel 114. All of the cross slots may be standardized so that any cross slot can receive the rail or the blade. The anchor blade 116 may be outboard of the rail 118, as shown in FIG. 1, or inboard.

In another example, the variable may be the height of the intermediate portion 117 of the anchor 112 or, in other words, the spacing between the rail portion and the blade portion. In this example, the anchor blade portion 116 may consistently lie outboard of the body 102. This arrangement may be appropriate in situations where the intermediate portion 117 and/or the rail portion 118 are long enough to protrude from the bottom surface 106 of the body 102 to contribute to overall bone fixation by the anchor 112, particularly when the protruding portions of the rail 118 and/or intermediate portion 117 include bone fixation features such as texturing, porous coating, osteoactive properties, and the like. This arrangement may also be appropriate in situations where the rail portion is located at a trailing end of an anchor (based on the anchor insertion direction), and a second blade portion is formed between the trailing rail portion and a leading end of the anchor, although this anchor embodiment may be used with the other arrangements disclosed herein.

The angle at which the blade 116 extends from the body 102 may vary to accommodate various joint anatomies. The cross slot 110 may hold the fully engaged anchor 112 at a fixed angle relative to the body 102. For example, the cross slot 110 may extend through the body 102 parallel to the body center axis, or at an acute angle to the body center axis. Where multiple channels and/or cross slots are present, the individual cross slots may be angled with respect to the body center axis so that the array of fully engaged anchors protrudes from the bottom surface 106 and converges, is parallel, or diverges. Other angular arrangements are contemplated for multi-anchor applications, such as a quasi-helical arrangement, bilateral convergence/divergence, or multi-point convergence/divergence.

In use, the anchors 112 may be secured to the body and driven into the glenoid to achieve fixation. These steps may occur in the stated order, a reverse order, or simultaneously.

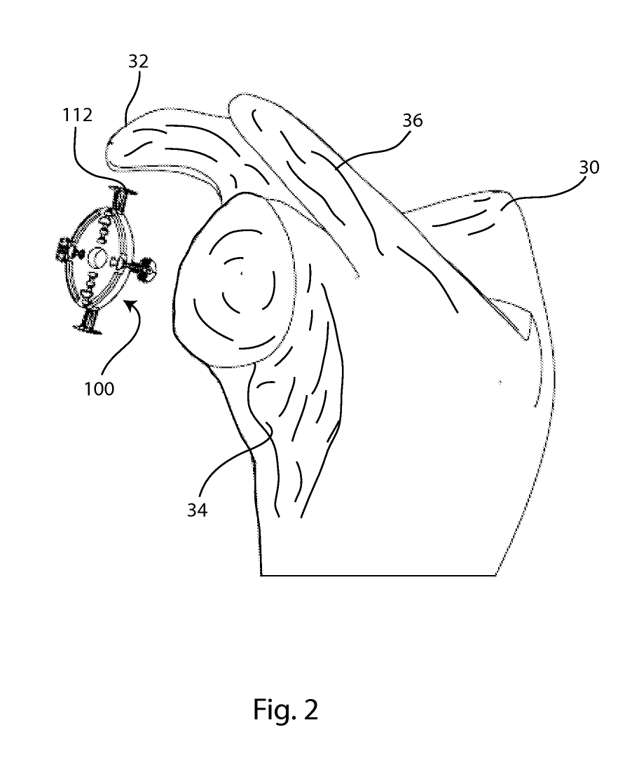

FIG. 2 illustrates the glenoid fixation device 100 positioned to be inserted into a glenoid vault. At least one anchor 112 may be inserted into one or more of the following bones: the coracoid process 30, the acromion process 32, the glenoid 34, and the clavicle 36. FIG. 2 illustrates an arrangement in which the channels 114 are formed into the body 102 along anatomical reference directions, axes, or planes. The channels 114 are aligned with anterior-posterior and superior-inferior directions. The channels 114 may optionally be aligned with major and minor dimensions of the glenoid socket regardless of anatomical reference directions.

The components disclosed herein may be made from metals, polymers, ceramics, glasses, composite materials, biological materials or tissues, or other biocompatible materials. Different materials may be used for individual components. Different materials may be combined in a single component.

It should be understood that the present system, kits, apparatuses, and methods are not intended to be limited to the particular forms disclosed. Rather, they are to cover all combinations, modifications, equivalents, and alternatives falling within the scope of the claims.

The claims are not to be interpreted as including means-plus- or step-plus-function limitations, unless such a limitation is explicitly recited in a given claim using the phrase(s) “means for” or “step for,” respectively.

The term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

In the foregoing Detailed Description, various features are grouped together in several embodiments for the purpose of streamlining the disclosure. This method of disclosure is not to be interpreted as reflecting an intention that the embodiments of the invention require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

1. A system comprising:
a body comprising a first surface, an opposite second surface, and a first channel, wherein the first channel extends through the first and second surfaces, wherein the first channel comprises a discrete first enlargement of a width of the first channel; and
a first anchor coupled to the body, wherein the first anchor comprises a first rail portion, a first blade portion, and a first intermediate portion connecting the first rail and first blade portions together, wherein the first rail portion and the first enlargement have complementary interlocking shapes, wherein the first rail portion is fixed within the first enlargement, the first intermediate portion occupies at least a portion of the first channel, and the first blade portion extends from the first surface of the body.

2. The system of claim 1, wherein the first channel comprises a second discrete enlargement of the width of the first channel, wherein the first and second enlargements are periodically spaced along a length of the first channel, wherein the first rail portion and the second enlargement have complementary interlocking shapes.

3. The system of claim 2, wherein the second enlargement is shaped to receive the first blade portion.

4. The system of claim 1, wherein the body comprises a second channel spaced apart from the first channel, wherein the second channel comprises a third discrete enlargement of a width of the second channel.

5. The system of claim 4, wherein the first rail portion and the third enlargement have complementary interlocking shapes.

6. The system of claim 4, comprising:
a second anchor coupled to the body, wherein the second anchor comprises a second rail portion, a second blade portion, and a second intermediate portion connecting the second rail and second blade portions together, wherein the second rail portion and the third enlargement have complementary interlocking shapes, wherein the second rail portion is fixed within the third enlargement, the second intermediate portion occupies at least a portion of the second channel, and the second blade portion extends from the first surface of the body.

7. A system comprising:
a body, wherein the body comprises a first surface, an opposite second surface, and a first connection feature; and
a first anchor coupled to the body at the first connection feature so that a portion of the first anchor protrudes from the first surface.

8. The system of claim 7, comprising:
a second anchor;
wherein the body comprises a second connection feature;
wherein the second anchor is coupled to the body at the second connection feature so that a portion of the second anchor protrudes from the first surface.

9. The system of claim 8, wherein the first and second anchors are interchangeably couplable to the body at the first and second connection features.

10. The system of claim 7, wherein the first and second anchors each comprise a blade portion, a rail portion, and an intermediate portion connecting the rail and blade portions together, wherein the rail portions of the first and second anchors are coupled to the body at the first and second connection features, respectively, and the blade portions of the first and second anchors protrude from the first surface.

11. The system of claim 10, wherein the blade portions of the first and second anchors converge.
